(19) United States
(12) Patent Application Publication
(10) Pub. No.: US 2010/0278900 A1
(43) Pub. Date: Nov. 4, 2010

(54) CHLORINE DIOXIDE BASED GUM AND CANDY

(75) Inventors: Heinz Peter Plaumann, Brownstown, MI (US); Christopher R. Castellano, Ringoes, NJ (US)

Correspondence Address: BASF CORPORATION, 100 Campus Drive, Florham Park, NJ, 07932, US

(73) Assignee: BASF Corporation

(21) Appl. No.: 12/772,525

(22) Filed: May 3, 2010

Related U.S. Application Data
(60) Provisional application No. 61/174,581, filed on May 1, 2009.

(51) Int. Cl. A61K 33/00; A61K 9/00; A61P 31/00

(52) U.S. Cl. 424/440; 424/600; 424/400

(57) ABSTRACT

Provided are oral compositions and devices comprising an active agent that is one of chlorine dioxide-generating components, a chlorine dioxide-containing solution, and combinations thereof.

### CROSS-REFERENCE TO RELATED APPLICATIONS

This application claims the benefit pursuant to 35 U.S.C. §119(e) of U.S. Provisional Application No. 61/174,581, filed on May 1, 2009, which is hereby incorporated by reference in its entirety herein.

### BACKGROUND

Infections of the oral cavity, such as of the tissue supporting teeth, are a common problem in mammals, including humans. Oral cavity infections range from caries development, arising from dental plaque bacterial damage to hard tooth tissue, to halitosis, arising from the volatile sulfur compounds (VSCs) produced by bacterial metabolic degradation of organic substances, to gingivitis, the inflammation of the gingiva (i.e., gum tissue) caused by dental plaque, which can progress to periodontis, a family of inflammatory diseases of periodontium. Current prophylactic and therapeutic treatments for oral tissue infections include brushing, flossing, topical fluoride, scaling and root planing, antiseptic mouth rinse, such as with a peroxide, antibiotics and, in extreme cases, surgical excision of infected tissue.

Efforts to whiten teeth have a long history, thought to date back to the Ancient Egyptians. The normal shade of teeth is determined by the natural off-white tints of the enamel and the dentin beneath. Extrinsic and intrinsic staining also contribute to tooth color. Extrinsic staining refers to surface stains, such as those caused by tea, coffee, red wine, and other foods rich in polyphones. Extrinsic stains are removed through the use of surfactants and/or abrasives, which cause their physical removal from the tooth surface. Intrinsic staining refers to stains that exist below enamel surface, or that penetrate below enamel surface. Removal of intrinsic staining is more difficult and time consuming than removal of extrinsic staining. Intrinsic stain removal can be achieved by a variety of methods including use of peroxides or peroxide analogs, with or without chemical, light or heat activation, to bleach the stains.

Tooth whitening products are available over-the-counter and as professional services in a dentist's office. Over-the-counter products typically contain carbamide peroxide or hydrogen peroxide as the bleaching agent. These products have concentrations of up to 21% carbamide peroxide (equivalent to 7% hydrogen peroxide) or as much as 10% hydrogen peroxide. In-office treatments generally use hydrogen peroxide as the oxidizer, at concentrations of 15% or more, and typically in the 25 to 35% range. At these high concentrations, rubber dams, or liquid dams with proper suction, must be used to prevent gingival irritation and ingestion.

Chlorine dioxide is known to be a disinfectant, as well as a strong oxidizing agent. The bactericidal, algaecidal, fungicidal, bleaching, and deodorizing properties of chlorine dioxide are also well known. These properties of chlorine dioxide have led to development of therapeutic and cosmetic applications for chlorine dioxide. For example, U.S. Publication No. 2009/0016973 describes the use of stabilized chlorine dioxide solutions for the prevention of oral disease. U.S. Pat. No. 5,281,412 describes chlorite and chlorine dioxide compositions that provide antiplaque and antigingivitis benefits without staining the teeth. Conventional methods of making chlorine dioxide, however, are not readily adaptable to over-the-counter formulations for such applications. For instance, the compositions described in U.S. Pat. No. 5,281,412 rely on a two-phase formulation to avoid premature reaction of ingredients. Compositions and devices for delivering chlorine dioxide to the oral cavity to contribute to oral care in a convenient, cost-effective way would satisfy a need in the art.

### SUMMARY

The following embodiments meet and address these needs. The following summary is not an extensive overview. It is intended to neither identify key or critical elements of the various embodiments, not delineate the scope of them.

Provided is an oral care composition comprising a) an active agent selected from the group consisting of chlorine dioxide-generating components, a chlorine dioxide-containing solution, and combinations thereof; and b) an orally acceptable carrier, wherein the oral care composition provides an efficacious amount of chlorine dioxide in an oral cavity. In an embodiment, the active agent can be compounded with the orally acceptable carrier. In another embodiment, the active agent is substantially encapsulated by the orally acceptable carrier.

In some embodiments, the active agent is chlorine dioxide-generating components. In some embodiments, the chlorine dioxide-generating components are in the form of a particulate precursor of chlorine dioxide.

In one embodiment, the active agent is a chlorine dioxide-containing solution. Optionally, the chlorine dioxide-containing solution is a substantially pure chlorine dioxide solution. Optionally, the chlorine dioxide-containing solution is a thickened fluid.

In some embodiments, the orally acceptable carrier comprises gum. In some embodiments, the active agent is coated with a wax coating.

Also provided is an oral cavity delivery device. The device comprises a first component comprising an active agent, the active agent being selected from the group consisting of chlorine dioxide-generating components, chlorine dioxide-containing solution, and combinations thereof; and a second component comprising an orally acceptable carrier, wherein the oral cavity delivery device can deliver an efficacious amount of chlorine dioxide in an oral cavity. On one embodiment, the second component forms a container comprising at least one hollow cavity and wherein the at least one hollow cavity contains the first component and substantially encapsulates the first component therein. In some embodiments, the container is substantially coated with a candy coating, a wax coating or both. In some embodiments, the first component is substantially encapsulated by a wax coating.

In some embodiments of the device, the active agent is chlorine dioxide-generating components. In some embodiments, the chlorine dioxide-generating components are in the form of a particulate precursor of chlorine dioxide.

In one embodiment, the active agent is a chlorine dioxide-containing solution. Optionally, the chlorine dioxide-containing solution is a substantially pure chlorine dioxide solution. Optionally, the chlorine dioxide-containing solution is a thickened fluid.

In some embodiments of the device, the orally acceptable carrier comprises gum.

### BRIEF DESCRIPTION OF THE DRAWINGS

For the purpose of illustrating the various compositions, devices, and methods, there are depicted in the drawings certain embodiments described herein. However, the compositions, devices and their methods of use are not limited to the precise arrangements and instrumentalities of the embodiments depicted in the drawings.

FIG. 1 is a schematic depicting the lamination of four gumsticks to create a delivery device comprising an interior cavity to contain an active agent. This depiction is merely exemplary and is not intended to limit the method of manufacturing such hollow-center delivery devices.

FIG. 2 is a schematic depicting the lamination of three gumsticks to create a delivery device comprising an interior cavity to contain an active agent. This depiction is merely exemplary and is not intended to limit the method of manufacturing such hollow-center delivery devices.

FIGS. 3A-3F depict schematics of exemplary embodiments comprising an active agent compounded in an orally acceptable carrier. The schematics are cross-sections to illustrate possible layers in such embodiments. The size and shape of these embodiments are not intended to be limiting.

FIGS. 4A-4F depict schematics of additional exemplary embodiments comprising an active agent and an orally acceptable carrier. The schematics are cross-sections to illustrate possible layers in such embodiments. The size and shape of these embodiments are not intended to be limiting.

FIG. 5 depicts a bar graph illustrating chlorine dioxide (in ppm) per gram of ASEPTROL® STab-2 for various gumball delivery device embodiments stored in different ways for about 18-19 hours prior to measurement of chlorine dioxide.

FIG. 6 depicts a bar graph illustrating chlorine dioxide (in ppm) per gram of ASEPTROL® STab-2 for various gumball delivery device embodiments stored in different ways for about 42 hours prior to measurement of chlorine dioxide.

FIG. 7 depicts a bar graph illustrating chlorine dioxide (in ppm) generated from gumball delivery devices comprising ASPETROL® STab-2 material in different forms. Gumballs were stored in different conditions for about 19 hours prior to measurement of chlorine dioxide.

FIG. 8 depicts a bar graph illustrating chlorine dioxide (in ppm) generated from gumstick delivery devices comprising ASPETROL STab-2 material in different forms. Gumsticks were stored in different conditions for about 23 hours prior to measurement of chlorine dioxide.

### DETAILED DESCRIPTION

Provided is an oral care composition and device containing an active agent selected from the group consisting of chlorine dioxide-generating components, a chlorine dioxide-containing solution, and combinations thereof; and an orally acceptable carrier. The oral care composition and device function as simple and easy-to-use delivery systems to deliver chlorine dioxide locally in the oral cavity. The delivered chlorine dioxide can have one or more efficacious effects including tooth whitening, reducing or eliminating mouth malodor, reducing or preventing plaque formation, alleviating or preventing periodontal disease, caries and other oral pathologies caused by pathogens.

### DEFINITIONS

Unless defined otherwise, all technical and scientific terms used herein generally have the same meaning as commonly understood by one of ordinary skill in the art. Generally, the nomenclature used herein and the laboratory procedures in cytopathicity analysis, microbial analysis, organic and inorganic chemistry, and dental clinical research are those well known and commonly employed in the art.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used. Generally, “about” encompasses a range of values that are plus/minus 10% of a reference value. For instance, “about 25%” encompasses values from 22.5% to 27.5%.

It is understood that any and all whole or partial integers between any ranges set forth herein are included herein.

As used herein, “biocidal” refers to the property of inactivating or killing pathogens, such as bacteria, algae, viruses, and fungi (e.g., anti-bacterial, anti-algal, antiviral and antifungal).

As used herein, an “efficacious amount” of an agent such as chlorine dioxide is intended to mean any amount of the agent that will result in a desired biocidal effect, a desired cosmetic effect, and/or a desired therapeutic biological effect in an oral cavity tissue. That result can be reduction and/or alleviation of the signs, symptoms, or causes of a disease in an oral cavity tissue. In one example, an efficacious amount of an agent is an amount that will result in whitening of a tooth with one or more treatments.

As used herein, a disease or disorder is “alleviated” if the severity of a symptom of the disease or disorder, the frequency with which such a symptom is experienced by a patient, or both, are reduced.

The term “chlorine dioxide-generating components” refers to at least an oxy-chlorine anion source and an acid source. Chlorine dioxide production is activated by contacting the chlorine dioxide-generating components with an aqueous fluid such as water or saliva and/or water vapor.

By “source of free halogen” and “free halogen source” is meant a compound or mixtures of compounds which release halogen upon reaction with water.

By “free halogen” is meant halogen as released by a free halogen source.

The term “particulate” is defined to mean all solid materials. By way of a non-limiting example, particulates may be interspersed with each other to contact one another in some way. These solid materials include particles comprising big particles, small particles or a combination of both big and small particles.

As used herein, a “particulate precursor of chlorine dioxide” refers to an intimate mixture of chlorine dioxide-forming components that are particulate. Granules of ASEPTROL®, ENLUXTRA® and CSR1.05F (BASF, Florham Park, N.J.) are exemplary particulate precursors of chlorine dioxide.

As used herein the term “acid source” refers to a material, usually a particulate solid material, which is itself acidic or produces an acidic environment when in contact with liquid water or solid oxy-chlorine anion.

As used herein, the term “source of free halogen” or “free halogen source” means a compound or mixtures of compounds which release halogen upon reaction with water. As used herein, the term “free halogen” means halogen as released by a free halogen source.

As used herein, “substantially pure chlorine dioxide solution” refers to a solution of chlorine dioxide that has a non-cytotoxic concentration of oxy-chlorine anion. As used herein, “substantially pure chlorine dioxide solution” also refers to a concentrated solution of chlorine dioxide that contains a concentration of oxy-chlorine anion that, upon dilution to an efficacious amount of chlorine dioxide, is not cytotoxic with respect to the concentration of oxy-chlorine anion.

As used herein, “oxy-chlorine anion” refers to chlorite (ClO₂⁻) and/or chlorate (ClO₃⁻) anions.

As used herein, “cytotoxic” refers to the property of causing lethal damage to mammalian cell structure or function. A composition is deemed “substantially non-cytotoxic” or “not substantially cytotoxic” if the composition meets the United States Pharmacopeia (USP) biological reactivity limits of the Agar Diffusion Test of USP <87>“Biological Reactivity, in vitro,” (approved protocol current in 2007) when the active pharmaceutical ingredient (API) is present in an efficacious amount.

By “substantially oxy-chlorine anion free chlorine dioxide composition” is meant a composition that contains an efficacious amount of chlorine dioxide and a non-cytotoxic and/or non-irritating concentration of oxy-chlorine anion, all as defined herein. The composition may contain other components or may consist essentially of oxy-chlorine anion free chlorine dioxide. The composition can be any type of fluid, including a solution or a thickened fluid, but can also be a gas or vapor comprising or consisting essentially of chlorine dioxide. The composition can be an aqueous fluid or a non-aqueous fluid.

As used herein, “irritating” refers to the property of causing a local inflammatory response, such as reddening, swelling, itching, burning, or blistering, by immediate, prolonged, or repeated contact. For example, inflammation of the gingival tissue in a mammal is an indication of irritation to that tissue. A composition is deemed “substantially non-irritating” or “not substantially irritating” if the composition is judged to be slightly or not irritating using any standard method for assessing mucosal irritation. Non-limiting examples of methods useful for mucosal irritation include: HET-CAM (hen's egg test-chorioallantoic membrane); slug mucosal irritation test; and in vitro tests using tissue-engineered oral mucosa or vaginal-ectocervical tissues. The skilled artisan is familiar with art-recognized methods of assessing mucosal irritation

The term “stable,” as used herein, is intended to mean that the components used to form chlorine dioxide, i.e., the chlorine dioxide-forming components, are not immediately reactive with each other to form chlorine dioxide. It will be understood that the components may be combined in any fashion, such as sequentially and/or simultaneously, so long as the combination is stable until such time that ClO₂ is to be generated.

The term “non-reactive,” as use herein, is intended to mean that a component or ingredient as used is not immediately reactive to an unacceptable degree with other components or ingredients present to form chlorine dioxide or mitigate the ability of any component or ingredient to perform its function in the formulation at the time of use. As the skilled artisan will recognize, the acceptable timeframe for non-reactivity will depend upon a number of factors, including how the formulation is to be formulated and stored, how long it is to be stored, and how the formulation is to be used. Accordingly, the timeframe for “not immediately reactive” will range from one or more minutes to one or more hours to one or more weeks. In one embodiment, the timeframe is a range of minutes, for instance, from one minute to about 60 minutes. In another embodiment, the timeframe is a range of hours, for instance, from about one hour to about 24 hours. In yet another embodiment, the timeframe is a range of days, for instance, from about one day to about one week. In yet another embodiment, the timeframe is a range of weeks, for instance, from about one week to about 4-6 weeks.

As used herein, “substantially encapsulate” means that a first material is enclosed within at least a second material, such that the entire surface of a first material is not exposed to ambient atmosphere. Substantial encapsulation of a first material can be obtained by a single material or by two or more materials such as a two part capsule, wherein each part is made of a different material.

As used herein, “tooth whitening” refers to a lightening of tooth shade relative to the tooth shade prior to treatment. Lightening can be assessed on an isolated or an in situ tooth by standard, art-recognized methods of assessing tooth shade, which include qualitative, quantitative and semi-quantitative methods. For instance, lightening can be assessed by simple visual inspection, e.g., by comparing “before” and “after” photographs of the treated teeth. Alternatively, a tooth can be deemed whitened when the tooth shade relative to the tooth shade prior to treatment is two or more shades lighter, as assessed by Vita classical shade guide (preferably under controlled visible light conditions) or two or more levels as assessed using the Vita Bleachedguide 3D-MASTER Shade system, which utilizes a multiple color spectrophotometer and includes half lightness levels. A difference of one shade is referred to herein as a “shade value unit” (SVU). Thus, for example, a difference of two shades is a 2 SVU difference.

As used herein, the phrase “oxidizing agent” refers to any material that attracts electrons, thereby oxidizing another atom or molecule and thereby undergoing reduction. Exemplary oxidizing agents include chlorine dioxide and peroxides, such as hydrogen peroxide.

As used herein, an “oral cavity infection” refers to a disease or disorder of a tissue in an oral cavity caused by a pathogenic infection. The pathogen may be bacterial, viral or fungal. A oral disease encompasses conditions wherein if the disease is not ameliorated then the animal's oral health continues to deteriorate. In contrast, an oral disorder is a state of oral health in which the animal is able to maintain homeostasis, but in which the animal's state of health is less favorable than it would be in the absence of the disorder. Left untreated, a disorder does not necessarily cause a further decrease in the animal's state of oral health. The term encompasses periodontal disease, halitosis, thrush and dental caries development.

As used herein, a “periodontal disease” is an infection of the tissues that support a subject's teeth, caused by a pathogenic infection. Periodontal disease includes gingivitis and periodontitis.

As used herein, “oral cavity tissue” refers to hard tooth tissue such as enamel and dentin and soft oral tissue such as oral mucosa and gums. Oral mucosa include buccal mucosa, other oral cavity mucosa (e.g., soft palate mucosa, floor of mouth mucosa and mucosa under the tongue) and the tongue.

As used herein, a “biofilm” refers to a biological aggregate that forms a layer on a surface, the aggregate comprising a community of microorganisms embedded in an extracellular matrix of polymers. Typically, a biofilm comprises a diverse community of microorganisms, including bacteria (aerobic and anaerobic), algae, protozoa and fungi. Monospecies biofilms also exist.

As used herein, “dental plaque” refers to a biofilm that forms on the surface of teeth.

As used herein, “hard tooth tissue” refers to at least one of enamel and dentin.

As used herein, “hard tooth tissue damage” refers to at least one of a reduction of microhardness of enamel, a reduction of microhardness of dentin, an increase in the surface roughness of enamel and an increase in the surface roughness of dentin.

As used herein, a composition “does not substantially damage hard tooth tissue” if one or more of the following is met for a tooth after treatment relative to the tooth prior to treatment: 1) enamel microhardness is decreased by an amount less than about 15% and/or the reduction is not statistically significant at the 5% confidence level; 2) dentin microhardness is decreased by an amount less than about 15% and/or the reduction is not statistically significant at the 5% confidence level; 3) enamel surface roughness is increased by an amount no more than about 20% and/or the increase is not statistically significant at the 5% confidence level; and 4) dentin surface roughness is increased by an amount no more than about 8% and/or the increase is not statistically significant at the 5% confidence level.

As used herein, an “orally acceptable carrier” refers to a material capable of encapsulating an active agent or being mixed or compounded with an active agent for delivery to the oral cavity, wherein the material does not cause harm to one or more oral cavity tissues of warm-blooded animals, such as humans.

The phrase “thickened fluid composition” encompasses compositions which can flow under applied shear stress and which have an apparent viscosity when flowing that is greater than the viscosity of the corresponding aqueous chlorine dioxide solution of the same concentration. This encompasses the full spectrum of thickened fluid compositions, including: fluids that exhibit Newtonian flow (where the ratio of shear rate to shear stress is constant and independent of shear stress), thixotropic fluids (which require a minimum yield stress to be overcome prior to flow, and which also exhibit shear thinning with sustained shear), pseudoplastic and plastic fluids (which require a minimum yield stress to be overcome prior to flow), dilantant fluid compositions (which increase in apparent viscosity with increasing shear rate) and other materials which can flow under applied yield stress.

A “thickener component,” as the phrase is used herein, refers to a component that has the property of thickening a solution or mixture to which it is added. A “thickener component” is used to make a “thickened fluid composition” as described above.

Unless otherwise indicated or evident from context, preferences indicated above and herein apply to the entirety of the embodiments discussed herein.

### DESCRIPTION

Provided is an oral care composition and an oral care device containing an active agent selected from the group consisting of chlorine dioxide-generating components, a chlorine dioxide-containing solution, and combinations thereof; and an orally acceptable carrier. The composition and device serve as delivery vehicles in the form of gum such as chewing gum and bubble gum and confectionaries such as hard and soft candies, lollipops and the like. As shown herein, representative embodiments of the oral care device comprising a chlorine dioxide-containing solution have been demonstrated to retain chlorine dioxide, thus enabling its delivery when desired. As further shown herein, oral care devices comprising chlorine dioxide-generating components have been demonstrated to retain substantial chlorine dioxide generating capacity after storage in various conditions and lengths of time.

Accordingly, the composition and the device can deliver an efficacious amount of chlorine dioxide in an oral cavity. Advantageously, chlorine dioxide has no significant taste issues at concentrations where it can function as a tooth whitening agent or as a biocidal agent. Moreover, chlorine dioxide has been found to not substantially damage hard tooth tissue. See, e.g., commonly-assigned US Pub. No. 2010/0015067. The composition and the device therefore provide a convenient, cost-effective solution for delivering chlorine dioxide to the oral cavity and thereby benefit from the many properties of chlorine dioxide. Therefore, use of the composition and/or the device is envisioned to contribute to tooth whitening, reducing halitosis (malodor), reducing plaque formation, reduce already-formed plaque, alleviating a periodontal disease, alleviating thrush, and/or reducing dental caries development. Use of the composition and/or the device is also envisioned to have a prophylactic effect, whereby the risk of developing an oral cavity infection is decreased.

### Active Agent

The active agent in the oral care composition or device can comprise chlorine dioxide-generating components. Chlorine dioxide-generating components refers to at least an oxy-chlorine anion source and an acid source. Exemplary oxy-chlorine anion sources include chlorites and chlorates and in particular, metal chlorites and metal chlorates. The metal chlorite or metal chlorate can generally be any metal chlorite or metal chlorate. In some embodiments, the metal chlorite is an alkali metal chlorite, such as sodium chlorite and potassium chlorite. Alkaline earth metal chlorites can also be employed. Examples of alkaline earth metal chlorites include barium chlorite, calcium chlorite, and magnesium chlorite. In many embodiments, the metal chlorite is sodium chlorite. Alkali metal chlorate and alkaline earth metal chlorate can also be employed as an oxy-chlorine anion source. The acid source may include inorganic acid salts, salts comprising the anions of strong acids and cations of weak bases, acids that can liberate protons into solution when contacted with water, organic acids, and mixtures thereof. In another aspect, the acid source in particular applications of the composition is a particulate solid material which does not react substantially with the metal chlorite during dry storage, however, does react with the metal chlorite to form chlorine dioxide when in the presence of the aqueous medium. The acid source may be water soluble, substantially insoluble in water, or intermediate between the two. Exemplary acid sources are those which produce a pH of below about 7, and below about 5.

Exemplary substantially water-soluble, acid-source-forming components include, but are not limited to, water-soluble solid acids such as boric acid, citric acid, tartaric acid, water soluble organic acid anhydrides such as maleic anhydride, and water soluble acid salts such as calcium chloride, magnesium chloride, magnesium nitrate, lithium chloride, magnesium sulfate, aluminum sulfate, sodium acid sulfate (NaHSO₄), sodium dihydrogen phosphate (NaH₂PO₄), potassium acid sulfate (KHSO₄), potassium dihydrogen phosphate (KH₂PO₄), and mixtures thereof. Other exemplary acid-source-forming components include dry solid hydrophilic materials such as synthetic zeolites; natural zeolites; hydrous clays; calcined clays, such as metakaolin, spinel phase kaolin, calcined bentonite, calcined halloysite, and calcined attapulgite; acidified synthetic zeolites; acidified natural zeolites; acidified clays; acidified calcined clays, and mixtures thereof. In some embodiments, the acid-source-forming component is sodium acid sulfate (sodium bisulfate). In other embodiments, the acid-source-forming component is calcined kaolin such as calcined kaolin microspheres or acidified calcined kaolin microspheres. Additional water-soluble, acid-source-forming components will be known to those skilled in the art.

In some embodiments, the chlorine dioxide-forming components optionally further comprises an energy-activatable catalyst. Exemplary catalysts are disclosed, for instance, in commonly-assigned International Patent Application No. PCT/US09/60580, filed Oct. 14, 2009. Exposure of the energy-activatable catalyst to electromagnetic energy, such as visible or ultraviolet light, results in the production of chlorine dioxide from the oxy-chlorine anion source. In other embodiments, the chlorine dioxide-forming components exclude an energy-activatable catalyst.

In some embodiments, the chlorine dioxide-forming components further comprise a free halogen source. In one embodiment, the free halogen source is a free chlorine source, and the free halogen is free chlorine. Suitable examples of free halogen source used in the anhydrous compositions include dichloroisocyanuric acid and salts thereof such as NaDCCA, trichlorocyanuric acid, salts of hypochlorous acid such as sodium, potassium and calcium hypochlorite, bromochlorodimethylhydantoin, dibromodimethylhydantoin, and the like. The exemplary source of free halogen is NaDCCA.

In some embodiments, the chlorine dioxide-generating components are in the form of a mixture. In these embodiments, the components are combined so as to be non-reactive. For instance, the combination can comprise at least one stabilizing component, for the purpose of preventing the reaction or degradation of one or more active components prior to the intended use of the composition. Stabilizing components are well known in the art. See, for instance, U.S. Pub. No. 2010/0015066.

In some embodiments, the chlorine dioxide-forming components are in the form of a particulate precursor of chlorine dioxide, which refers to an intimate mixture of chlorine dioxide-forming components that are particulate. An intimate mixture generally means the various component particles are interspersed and in contact with each or are able to be in contact with each. The distribution of the different component particles can be substantially homogenous or partially homogenous. In these embodiments, the particulate precursor of chlorine dioxide is stable. Optionally, the particulate precursor further comprises one or more of magnesium chloride, a low solubility porous framework former, and a clay. Examples of these are found, for instance, in U.S. Pat. No. 6,699,404.

In the oral care composition or device, chlorine dioxide-generating components can be present as a one or a few massive bodies. As used herein, the term “massive body” means a porous solid shape, comprising a mixture of granular particulate ingredients, wherein the size of the particles comprising the ingredients is substantially smaller than the size of the massive body. Such massive bodies may be formed by a variety of means known in the art, such as tableting, briquetting, extrusion, sintering, granulating and the like, such as a tablet. Alternative, the components can be in the form of granules. Such granules can be formed by crushing a massive body, or compacting a mixture comprising or consisting of chlorine dioxide-generating components.

In an embodiment, the particulate precursor is an ASEPTROL®, ENLUXTRA® or CSR1.05F (BASF Corporation, Florham Park, N.J.) product. In some embodiments, the particulate precursor is ASEPTROL® S-Tab2. ASEPTROL S-Tab2 has the following chemical composition by weight (%): NaClO₂ (7%); NaHSO₄ (12%); NaDCC (1%); NaCl (40%); MgCl₂ (40%). Example 4 of U.S. Pat. No. 6,432,322 describes an exemplary manufacture process of S-Tab2. S-Tab2 is manufactured as a tablet. Such tablets can be used whole, or can be broken into a few smaller pieces. Granules can be produced, either by comminuting pressed S-Tab2 tablets, or by dry roller compaction of the non-pressed powder of the S-Tab2 components, followed by breakup of the resultant compacted ribbon or briquettes, and then screening to obtain the desired size granule. Upon exposure to water or an aqueous fluid, chlorine dioxide is generated from the ASEPTROL® granules. In another embodiment, the particulate precursor is an ASEPTROL® controlled sustained release formulation, such as CSR1.05F. CSR1.05F consists of about 93.75 wt. % calcined kaolin microspheres and 6.25 wt. % technical grade NaClO₂ (80% purity). CSR1.05F is exemplary of materials described in commonly-assigned U.S. Pat. No. 6,294,108.

In the composition or device, the chlorine dioxide-generating components can be in the form of a substantially dry material. Alternatively, the chlorine dioxide-generating components can be mixed or suspended in a liquid that does not substantially activate chlorine dioxide generation prior to contact with the activator, such as saliva.

The active agent in the oral care composition or device can also be a chlorine dioxide-containing solution. Preparation of a chlorine dioxide-containing solution is well known in the art. In some embodiments, the chlorine dioxide-containing solution is a substantially oxy-chlorine anion free chlorine dioxide solution. Advantageously, such a solution is substantially non-cytotoxic and/or non-irritating. Methods for preparing substantially oxy-chlorine anion free chlorine dioxide solution are disclosed in commonly-assigned U.S. Pub. No. 2010/0015066, incorporated herein by reference in its entirety.

In some embodiments, droplets of a chlorine dioxide-containing solution are surrounded by a coating to produce encapsulated particles including but not limited to microcapsules. Such particles can then be use to prepare the oral care compositions and devices described herein. Methods of preparing particles of encapsulated liquids are known in the art. The coating material for such encapsulated particles can protect the contents premature loss of chlorine dioxide by diffusion and/or interaction with the orally acceptable carrier.

In some embodiments, the chlorine dioxide-containing solution is a thickened solution. Thickening agents are well known in the art and can be categorized into four groups: natural hydrocolloids (also referred to as “gum”), semisynthetic hydrocolloids, synthetic hydrocolloids, and clay. Exemplary thickeners suitable for the oral care composition include, but are not limited to, carboxymethylcelluloses. In one embodiment, the thickener is sodium carboxymethylcellulose.

Methods of preparing substantially non-cytotoxic thickened compositions comprising chlorine dioxide are also disclosed in commonly-assigned U.S. application Ser. Nos. 12/502,326 and 12/502,356, filed Jul. 14, 2009, entitled “Non-Cytotoxic Chlorine Dioxide Fluids,” incorporated herein by reference in their entirety. The presence of oxy-chlorine anions in a composition comprising chlorine dioxide can lead to cytotoxicity. Accordingly, in some embodiments, the chlorine dioxide-containing solution has a non-cytotoxic amount of oxy-chlorine anions. A composition comprising chlorine dioxide that comprises from zero milligram (mg) oxy-chlorine anion per gram composition to no more than about 0.25 mg oxy-chlorine anion per gram composition, from zero to 0.24, 0.23, 0.22, 0.21, or 0.20 mg oxy-chlorine anion per gram composition, from zero to 0.19, 0.18, 0.17, 0.16, 0.15, 0.14, 0.13, 0.12, 0.11, or 0.10 mg oxy-chlorine anion per gram composition and from zero to 0.09, 0.08, 0.07, 0.06, 0.05 or 0.04 mg oxy-chlorine anion per gram composition, absent other constituents that contribute to cytotoxicity, is substantially non-cytotoxic.

Soft tissue irritation can result from highly reactive oxygen species and/or extremes of pH, both acidic and basic. To minimize soft tissue irritation by the chlorine dioxide-containing solution, the solution has a pH of at least 3.5. Preferably, the solution has a pH of at least 5, and more preferably still, greater than about 6. In certain embodiments, the pH ranges from about 4.5 to about 11, more preferably from about 5 to about 9, and more preferably still, greater than about 6 and less than about 8. In one embodiment, the pH is about 6.5 to about 7.5. The concentration of oxy-chlorine anions is not believed to be a primary contributor to mucosal tissue irritation.

The oral care compositions and oral care devices are intended to deliver to an oral cavity a quantity of chlorine dioxide to contribute to one or more of tooth whitening, reduction of halitosis (malodor), reduction of plaque formation, reduction of already-formed plaque, alleviation of a periodontal disease, alleviation of thrush, and/or reduction dental caries development such as reduction in the rate of development or the number of caries developed. Suitable ranges of chlorine dioxide to deliver range from about 1 part-per-million (ppm) to about 1000 ppm, about 1 ppm to about 600 ppm, about 5 ppm to about 500 ppm or about 5 ppm to about 250 ppm. The skilled artisan can readily determine the amount of chlorine dioxide or chlorine dioxide-generating components are needed to incorporate into a single portion of the composition or a single device. Non-limiting amounts of an exemplary particulate precursor such as ASEPTROL® Stab-2 to include in a single portion or device range from about 0.01 grams to about 10 grams, from about 0.1 grams to about 5 grams, and from about 1.0 grams to about 3 grams.

### Orally Acceptable Carrier

Orally acceptable carriers are well known to the skilled artisan. Exemplary orally acceptable carriers include, but are not limited to, gums and confectionary material such as soft and hard candies. Gums include but are not limited to polyisobutylene, butadiene-based synthetic rubbers such as styrene-butadiene rubber, natural gums such as chicle, jelutong, balata, crown gum, gutta-percha, sorva and the like, butadiene-styrene copolymer, isobutylene-isoprene copolymer, polyethylene, and the like, and mixtures thereof. Confectionary material encompasses soft and hard formulations. Confectionary materials suitable for preparing a lozenge, tablet, pastille, caramel or other confectionary delivery form are well known in the art. Typical ingredients include, but are not limited to, sugar, corn syrup, colors, flavorants, and bulking agents. Formulations excluding sugar and corn syrup are also known in the art.

Another exemplary carrier is a wax, such as beeswax, carnauba wax, paraffin wax, or combinations thereof, that can be shaped so as to fully encapsulate a chlorine dioxide-containing solution. This type of device is similar to so-called syrup filled wax bottles. The shape of a wax container filled with a chlorine dioxide-containing solution, however, is not limited to a bottle shape. The wax can be mixed with other compounds, such as oils, to modify its formability.

### Optional Components

The composition can optionally comprise one or more other components. Such components include, but are not limited to, sweeteners, flavorants, coloring agents, and fragrances. Sweeteners include sugar alcohols. Exemplary sugar alcohols include sorbital, xylitol, lactitol, mannitol, maltilol, hydrogenated starch hydrolysate, erythritol, reducing paratinose, and mixtures thereof. Flavoring agents include, e.g., natural or synthetic essential oils, as well as various flavoring aldehydes, esters, alcohols, and other materials. Examples of essential oils include oils of spearmint, peppermint, wintergreen, sassafras, clove, sage, eucalyptus, marjoram, cinnamon, lemon, lime, grapefruit, and orange. Coloring agents include a colorant approved for incorporation into a food, drug, or cosmetic by a regulatory agency, such as, for example, FD&C or D&C pigments and dyes approved by the FDA for use in the United States. Fragrances include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA), and the like.

Other optional components include: antimicrobial agents such as antibacterial agents and antifungal agents, enzymes, malodor controlling agents, cleaning agents such as phosphates, antigingivitis agents, antiplaque agents, antitartar agents, anticaries agents such as a source of fluoride ion, antiperiodontitis agents, nutrients, antioxidants, remineralizing agents such as casein phosphopeptide-amorphous calcium phosphate, and the like. Exemplary antimicrobial agents include but are not limited to: doxycycline, metronidazole, chlorhexidine, minocycline and tetracycline. Exemplary antifungal agents include, but are not limited to, miconazoel, nystatin and amphotericin. Yet other optional components include elastomers, elastomer solvents, waxes, emulsifiers, plasticizers, softeners, surfactants, and dispersing agents. Such components are well known in the art. See, e.g., U.S. Pat. No. 6,696,044 and U.S. Pub. No. 2010-0061941. In some embodiments, an abrasive can be included to aid in stain reduction. In other embodiments, abrasives are excluded. Chlorine dioxide has been demonstrated to have a polishing effect in the absence of traditional abrasives. See, e.g., commonly-assigned U.S. Pub. No. 2010-0015251. In some embodiments, the composition can comprise an additional oxidizing agent(s)ₜ. In other embodiments, a second oxidizing agent is excluded.

It is preferred that all optional components are relatively resistant to oxidation by chlorine dioxide, since oxidation of composition components by chlorine dioxide will reduce the available chlorine dioxide for oxidation for its intended function. “Relatively resistant” means that in the time scale of preparing and using the chlorine dioxide-containing composition in an application, the function of the optional component is not unacceptably diminished, and the composition retains an acceptable level of efficacy/potency with respect to the chlorine dioxide. In some embodiments, the optional components comprise or consist of compatible additives. A compatible additive is a compound that is substantially non-reactive with chlorine dioxide. Compatible additives are described in commonly-assigned U.S. provisional application 61/299,999, filed Jan. 21, 2010, incorporated herein in its entirety. Exemplary compatible additives include L-menthol, benzaldehyde, camphor, methone, ethyl menthane carboxamide, eucalyptol, aspartame, sodium cyclamate, sodium saccharin dihydrate, sucralose, cetylpyridinium chloride, benzoic acid, benzoyl alcohol, propylene glycol, polyethylene glycol 400, glycerol, ethyl acetate, ethanol, hydroxypropyl methylcellulose, acetone, glycine, 1-glutamic acid, boric acid, citric acid and sodium bisulfate.

### Formulations and Devices

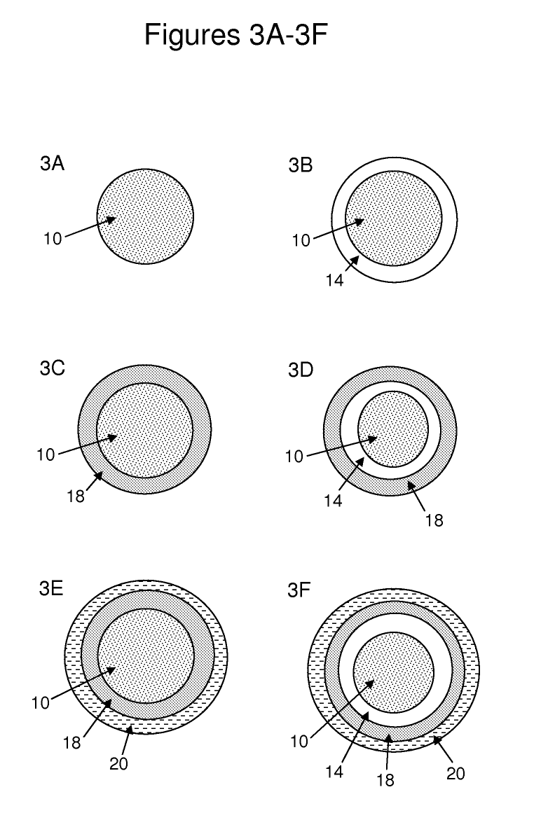

The oral care composition can formulated in a variety of ways. In one aspect, the oral care composition comprises chlorine dioxide-generating components or a chlorine dioxide-containing solution mixed or compounded in an orally acceptable carrier. In these embodiments, it is contemplated that droplets of the chlorine dioxide-containing solution can be surrounded by a coating to produce encapsulated particles such as microcapsules, and these particles can be compounded with an orally acceptable carrier. FIG. 3A depicts an exemplary representation of active agent mixed or compounded in an orally acceptable carrier 10. In one embodiment, the composition can be coated. For instance, as depicted in FIG. 3B, a portion of the chlorine dioxide-generating components or encapsulated chlorine dioxide-containing solution particles compounded in an orally acceptable carrier 10 can be substantially entirely coated 14 with additional orally acceptable carrier that does not comprise active agent. The additional carrier can be the same orally acceptable carrier comprising active agent, or it can be different. In another embodiment, the composition is coated to produce a hard or crunchy candy coating. FIG. 3C depicts active agent compounded in an orally acceptable carrier 10 coated with a hard or crunchy candy coating 18. FIG. 3D depicts the composition as shown in FIG. 3B further comprising the candy coating 18. Such a coating typically comprises a sweetener, a flavorant and a coloring agent. In yet another embodiment, the composition is coated with a wax or wax-containing material. FIG. 3E depicts active agent compounded in an orally acceptable carrier 10 coated with a hard or crunchy coating 18, further coated with a wax coating 20. FIG. 3F depicts the composition as shown in FIG. 3D further comprising the wax coating 20. As depicted in FIG. 3F, the oral care composition can comprise active agent compounded in an orally acceptable carrier 10, coated first with a hard or crunchy candy coating 18 and which is then overcoated with a layer of wax 20. A wax coating is advantageous to minimize contact of the chlorine dioxide-generating components with water vapor or water, which could contribute to premature chlorine dioxide generation, as well as to contribute to the integrity of the orally acceptable carrier. Thus, a wax coating can contribute to extending shelf life of the oral care composition or device. A wax coating can also be used to substantially coat a portion of the chlorine dioxide-generating components or encapsulated chlorine dioxide-containing solution particles compounded in an orally acceptable carrier 10. In this embodiment, the wax-coated active agent is optionally further encapsulated with additional orally acceptable carrier. Optionally, one or more candy coatings can overcoat the wax layer or the layer of additional orally acceptable carrier. Optionally, another wax coat is overcoated on the one or more candy coatings.

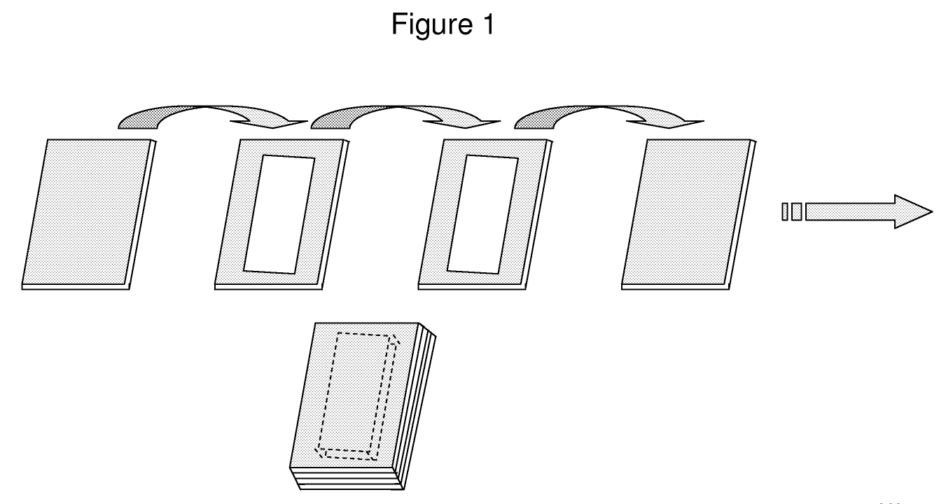

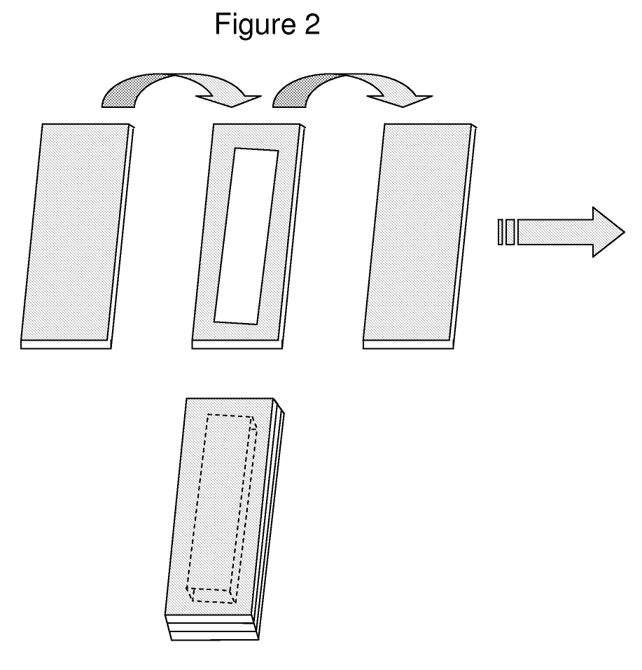

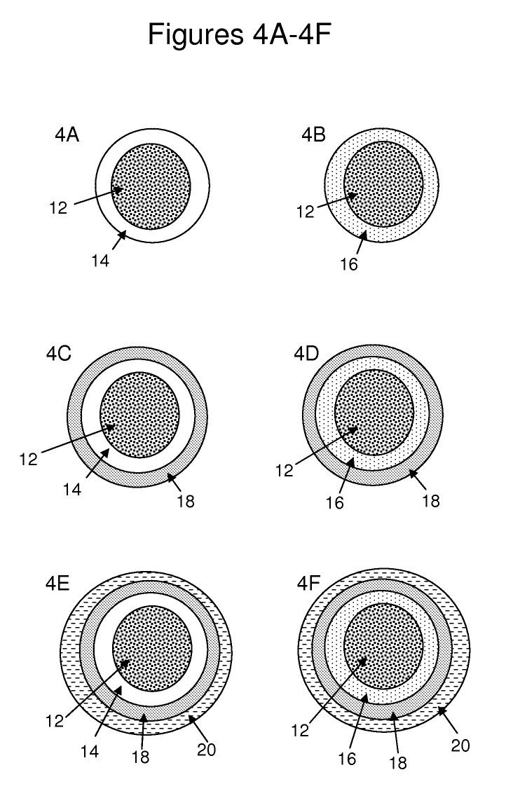

Similarly, the oral care device can have a wide variety of embodiments. In one aspect, an oral care device comprises the orally acceptable carrier as one component in a shape that includes a interior cavity or hollow portion. The active agent as another component is placed into the interior cavity or hollow portion, and is generally substantially encapsulated or surrounded by the carrier (or is otherwise protected from undesired exposure to water or water vapor). In one embodiment, the active agent in the interior cavity or hollow portion is coated with a wax layer, to aid in minimizing contact of the active agent with water, water vapor or atmosphere and contribute to shelf life, and can minimize or preclude contact with an orally acceptable carrier that can react with the active agent and undesirably consume chlorine dioxide or chlorine dioxide-generating components. The oral care device can be any shape including but not limited to gumball, sticks, pellets and the like. See, for instance, FIGS. 1 and 2. In another embodiment, FIG. 4A depicts a roughly spherical shape such as a gumball made of on orally acceptable carrier 14 wherein the hollow center of the sphere contains active agent 12. In some embodiments, the active agent 12 is a chlorine dioxide-containing solution, which is substantially encapsulated by the orally acceptable carrier, so as to minimize loss or leakage of the solution. Optionally, the chlorine dioxide-containing solution is first encapsulated in a material that is not substantially reactive with water such as a wax, which is then encapsulated with the orally acceptable carrier. In some embodiments, the active agent 12 is chlorine dioxide-generating components in a liquid that does not substantially activate chlorine dioxide generation, which is substantially encapsulated by the orally acceptable carrier 14 (FIG. 4A) or orally acceptable carrier compounded with additional active agent 16 (FIG. 4B), so as to minimize loss or leakage of the liquid. In some embodiments, the active agent 12 is chlorine dioxide-generating components in a dry form, such as tablets, granules or powder, which is substantially encapsulated by the orally acceptable carrier active agent 14 (FIG. 4A) or orally acceptable carrier comprising additional active agent 16 (FIG. 4B) to minimize premature contact with water or water vapor. As with the oral care composition, the oral care devices can comprise additional layers. For instance, as depicted in FIGS. 4C and 4D, the device can further comprise a hard or crunchy candy coating 18. Optionally, as depicted in FIGS. 4E and 4F, the candy-coated device further comprises a wax coating 20 on the exterior of the device.

The described formulations and devices are exemplary but not intended to be limiting. Additional embodiments of the oral care composition and oral care device will be readily apparent to the skilled artisan in view of this disclosure. Such embodiments are encompassed within the subject matter described and claimed.

### Manufacture and Use

The oral care composition and device can be manufactured using conventional methods known to the skilled artisan for preparing compounded materials and center-filled gums and center-filled confectionaries. Non-limiting examples of such manufacturing methods include U.S. Pat. Nos. 4,513,012; 4,656,039; 5,866,179; 6,479,071; 6,491,540; 6,696,044; U.S. Pub. No. 2010-0074988; and International Patent Publication WO 2006/026298. Methods of coating gum and confectionaries are similarly well known in the art and include, but are not limited to, pan coating and spray coating.

In manufacturing the oral care compositions and devices, care is taken to avoid exposing the metal chlorite in the chlorine dioxide-generating components to temperatures in excess of about 150° C. to minimize decomposition of chlorite. A temperature below about 150° C., preferably below about 135° C., and most preferably below about 110° C. is desirable for minimizing metal chlorite decomposition during the manufacturing process. Care is also taken to minimize contact between the chlorine dioxide-generating components and water or water vapor during manufacture. One of skill in the art will recognize that the extent of water and humidity control necessary will depend in part on the specific materials involved in the manufacturing and the manufacturing process itself. The skilled artisan can identify appropriate manufacturing conditions regarding temperature and water or humidity exposure for any particular composition or device through routine experimentation. It is known in the art that chlorite can react with organic materials, leading to potentially explosive conditions. The skilled artisan can identify appropriate manufacturing conditions to minimize the risk of such explosive conditions regarding a composition or device comprising an organic material through routine experimentation.

Once manufactured, the compositions and devices are preferably packaged and stored in such a way as to minimize exposure to water vapor, water or aqueous liquids prior to their use in oral care. In use, the compositions and devices generally deliver chlorine dioxide when the composition or device is placed in an oral cavity. The compositions and devices are intended to remain in the mouth for at least several minutes, for instance about 5 to about 60 minutes, about 5 to about 30 minutes, or about 10 to about 20 minutes. However, some individuals may and can keep the composition in the mouth for one or more hours if so desired. Embodiments in which the active agent is in the form of chlorine dioxide-containing solution generally have the active agent substantially completely encapsulate, for instance, with the orally acceptable carrier, and optionally one or more coatings. In these embodiments, chlorine dioxide is released into the oral cavity after the encapsulating material is penetrated, such as by a tooth, thereby permitting the chlorine dioxide-containing solution to exit the cavity and enter the oral cavity and to release chlorine dioxide therein.

In embodiments in which the active agent is in the form of chlorine dioxide-generating components, the active agent generally needs to be contacted with water such as saliva or water vapor such as breath in order to activate the acid source and the generation of chlorine dioxide. If the active agent is compounded in the orally acceptable carrier, chlorine dioxide generation should begin from the components at the surface of the composition, upon contact with saliva or breath. Biting or chewing the composition will enable chlorine dioxide generation from the components within the composition or device, as these components become contacted with saliva. In embodiments where the active agent is substantially encapsulated by the orally acceptable carrier and optional coatings, chlorine dioxide generation will begin upon penetration of the carrier and optional coatings, thereby exposing the chlorine dioxide-generating components to saliva and/or breath.

It is contemplated that use of the oral care compositions and delivery devices, and in particular regular use, can contribute beneficially to tooth whitening and oral hygiene, as discussed elsewhere herein.

The compositions are further described in detail by reference to the following experimental examples. These examples are provided for purposes of illustration only, and are not intended to be limiting unless otherwise specified. Thus, the compositions should in no way be construed as being limited to the following examples, but rather, should be construed to encompass any and all variations which become evident as a result of the teaching provided herein.

### EXAMPLES

This series of studies was designed to assess whether significant amounts of chlorine dioxide can be generated when active agent is incorporated into various chewing gum embodiments. Various embodiments were prepared and stored under different conditions for different periods of time. The amount of chlorine dioxide was then assessed.

The materials and methods used in the following examples are now described.

SAMPLE PREPARATION: Chewing gum embodiments tested included large gum balls as well as a lamination of chewing gum sticks forming a hollow vessel (see FIGS. 1 and 2). In addition, the active agent in these embodiments included: ASEPTROL® STab-2 tablets (375 mg per tablet), ASEPTROL® STab-2 granules, CSR1.05F powder, and various NaCMC gels thickened chlorine dioxide-containing solutions (e.g. sodium carboxymethylcellulose; NaCMC) gels containing chlorine dioxide. Test samples were prepared by weighing all of the raw materials on an analytical balance. Gumballs and gum sticks that were used were weighed before and after the addition of any chlorine dioxide solution or chlorine dioxide-generating components. The date and time that the ClO₂-generating material was added to the gum delivery vehicle was documented. The compositions of the various test samples were calculated from the masses recorded during raw material weighing.

The controls were prepared fresh as needed. NaCMC gel was prepared as follows: 7.2 g of NaCMC was added to 232.8 g USP water and allowed to hydrate overnight. Then, 120 g of a 600 ppm ClO₂ solution made from ASEPTROL® STab-2 was then added to the hydrated gel and mixed until homogeneous. For the “STab-2 tablet” control, one 375 mg STab-2 tablet was dissolved in 50.0 g of USP water. For the “STab-2 granules” control, 375 mg STab-2 granules (<100 mesh) was dissolved in 50.0 g of USP water. At least one control sample was made for every batch of chewing gum embodiments when possible and was prepared using the same method and containers that were used to prepare the samples.

ANALYTIC TESTS: Each test sample was tested for chlorine dioxide at a fixed time point, which occurred approximately 18 to 42 hours after addition of the ClO₂-generating material to the chewing gum embodiment. Chlorine dioxide in each test sample was generally assessed by one of two methods: absorption or titration. Titration was used for water-insoluble solid materials, such as CSR1.05F, which contains calcined kaolin. In a few cases, chlorine dioxide test strips were used.

I. Absorption Method: If a test sample was clear and free of any dispersed phase (e.g., cloudiness, precipitate, etc), the chlorine dioxide concentration was measured at 445 nm using the “75 Chlor Method” on the Hach DR2700 Portable Spectrometer (Serial #1319099, Hach Co., Loveland, Colo.). The instrument manufacturer calibrates the absorption of the spectrometer at 445 nm with chlorine dioxide standards. The readings obtained on the unit were then corrected by multiplying the reading by 0.856, and adding 3.93 to the result to yield the correct ClO₂ content in ppm.

For test samples that were analyzed by UV/Visible spectroscopy, the test sample was weighed on a balance, then reacted with 50.0 mL of USP H₂O. The sample was allowed to dissolve completely. After dissolution was complete, the resulting solution was swirled gently to ensure homogeneity. Then, 10.0 mL of the solution was pipetted into a clear Hach vial for assessment of chlorine dioxide.

II. Titration: If a test sample could not be measured by the UV/Vis absorption method due to turbidity from a dispersed phase, the concentration of chlorine dioxide in aliquots of the test sample was determined by reacting ClO₂ with an excess of potassium iodide at neutral pH to form iodine in aqueous solution. The iodine solution was then titrated with sodium thiosulfate solution using a starch indicator to a colorless endpoint. The titration was performed using a Hach test kit (Hach Co., Catalog #25711-00). Calculation of the chlorine dioxide concentration, assuming that no chlorine or other reactive oxidizers are present, is:

$$[ {ClO}_{2} ] = \frac{N \times Titer 7 \times M}{V} \qquad ( 1 )$$

[ClO₂]=Concentration of ClO₂ in solution, mg/liter

N=Normality of sodium thiosulfate solution, equivalents/liter

Titer7=Volume of sodium thiosulfate solution, ml

M=is the molar weight of ClO₂ in mg per mole

V=Volume of the aliquot, ml

III. ClO₂ Test Strips: For NaCMC samples where the available sample size was small, and it was determined by visual inspection of the sample that a low ClO₂ concentration was likely, ClO₂ test strips (Chlorine Dioxide Check; Part #480031, Lot #12028B; Industrial Test Systems, Inc., Rock Hill, S.C.) were used as per the label instructions to estimate the amount of ClO₂ present.

CONTAINERS: Samples were stored in one of three locations: inside a dry box purged with dry N₂ (“dry box” below); inside a closed drawer (“ambient” below); or inside a refrigerator. The date and time that a sample was transferred from the gum embodiment for analysis was recorded.

### Experiment 1

### Active Agent is Chlorine Dioxide-Containing Solution

The following are descriptions of three specific examples. These descriptions are representative of the preparation of other examples, as indicated.

The following description is representative of Examples 1-5.

Example 5: A 3.0 wt % NaCMC gel containing 140 ppm ClO₂ was prepared. A 60 cc plastic syringe was filled with this ClO₂ gel, and affixed with an 18-gauge needle. A large round gumball (Rayge Candy Co., Brick, N.J.) weighing 7.8474 g was filled with the aforementioned gel by piercing the shell and depressing the plunger. After filling, the gumball weighed 9.9782 g, indicating that the amount of gel inserted was 2.1308 g.

The gumball was then placed into a closed drawer under ambient conditions. After nearly 48 hours, the gumball was removed from the drawer. A ClO₂ test strip was then immersed into the gel after opening the gumball, which was found to contain 1 ppm ClO₂.

The following description is representative of Examples 6-10.

Example 9: A 3.0 wt % NaCMC gel containing 140 ppm ClO₂ was prepared. A 60 cc plastic syringe was filled with this ClO₂ gel, and affixed with an 18-gauge needle. A large round gumball (Rayge Candy Co., Brick, N.J.) weighing 7.8187 g was filled with the aforementioned gel by piercing the shell and depressing the plunger. After filling, the gumball weighed 10.3065 g, indicating that the amount of gel inserted was 2.4878 g.

The gumball was then placed into a heat-sealed bag, and placed into a refrigerator. After nearly 48 hours, the gumball was removed from the bag. A ClO₂ test strip was then immersed into the gel after opening the gumball, which was found to contain 5 ppm ClO₂.

The following description is representative of Examples 11-13.

Example 13: A 3.0 wt % NaCMC gel containing 140 ppm ClO₂ was prepared. A 60 cc plastic syringe was filled with this ClO₂ gel, and affixed with an 18-gauge needle. A stack of gum sticks (WM Wrigley Jr. Company, Chicago, Ill.) was arranged to form a vessel as depicted in FIG. 1. The gum stick vessel weighing 8.5522 g was filled with the aforementioned gel by inserting the needle between layers and depressing the plunger. After filling, the gum vessel weighed 10.6555 g, indicating that the amount of gel inserted was 2.1033 g.

The gum vessel was then placed into a heat-sealed bag and stored in a closed drawer. After nearly 48 hours, the gum vessel was removed from the bag. A ClO₂ test strip was then immersed into the gel after opening the gum stick vessel, which was found contain 0.5 ppm ClO₂.

The controls, C1-C4, were 3.0% NaCMC gel, prepared fresh as described above.

The data for Examples 1-13, in the order they were measured after about 48 hours storage, is presented in Table 1.

TABLE 1

| | Delivery | Storage | ClO₂ test | ClO₂ |
|---|---|---|---|---|
| Example # | device form | method | method | (ppm) |
| C1 | n/a | n/a | titration | 92.7 |
| C2 | n/a | n/a | titration | 123.1 |
| 3 | Gumball | Ambient | titration | <0.1 |
| 4 | Gumball | Ambient | titration | <0.1 |
| 2 | Gumball | Ambient | titration | <0.1 |
| 1 | Gumball | Ambient | titration | <0.1 |
| 8 | Gumball | Bag in refrigerator | titration | <0.1 |
| C3 | n/a | n/a | titration | 87.0 |
| 11 | Gumstick | Ambient | titration | <0.1 |
| 12 | Gumstick | Ambient | titration | <0.1 |
| 5 | Gumball | Ambient | test strip | 1 |
| 9 | Gumball | Bag in refrigerator | test strip | 5 |
| 13 | Gumstick | Bag in drawer | test strip | 0.5 |
| 6 | Gumball | Bag in drawer | test strip | 1 |
| 10 | Gumball | Bag in refrigerator | test strip | 7 |
| C4 | n/a | n/a | test strip | >100 |
| 7 | Gumball | Bag in drawer | test strip | 0.5 |

Substantially no chlorine dioxide was detected for Examples 1-4, 8, 11 and 12, which were largely stored at ambient conditions. Chlorine dioxide was detected for Examples 5-7, 9, 10 and 13. The gum samples not stored in a refrigerator were observed to become very pliable, which may have contributed to loss of chlorine dioxide. Without wishing to be bound by theory, it is believed that the sugar in the chewing gum reacted with water contained in the gel, and adversely affected the integrity of the gum. As a result, it is believed that the ClO₂ also either reacted with a gum ingredient or simply had a chance to dissipate prematurely to the atmosphere. The examples stored in the refrigerator were observed to have better integrity. Two of these examples retained a good amount of chlorine dioxide after storage. It was also observed that most of the Examples stored in heat-sealed bags retained a detectable quantity of chlorine dioxide.

These data suggest that oral care devices containing chlorine dioxide-containing solution can be prepared such that the device retains the chlorine dioxide during storage. Gums that are less reactive with water can be used. Alternatively or additionally, the chlorine dioxide-containing solution can be encapsulated in a material that is substantially not reactive with water and then this encapsulated active agent can be coated with a gum. Alternatively or additionally, the oral care devices can comprise a candy coating and optionally a wax coating to aid in maintaining the integrity of the device and retention of the chlorine dioxide in the chlorine dioxide-containing solution.

### Experiment 2

### Active Agent is Chlorine Dioxide-Generating Components

The following are descriptions of eight specific examples. These descriptions are representative of the preparation of other examples, as indicated. Examples 14-27 were stored for about 18-19 hours, then the chlorine dioxide was measured. Examples 28-39 were stored for about 42 hours prior to measurement of chlorine dioxide.

The following description is representative of Examples 14, 15, 28 and 29.

Example 14: A large round gumball (Dubble Bubble; Concord Confections, Ltd., Concord, Ontario, Canada) was placed onto a holder and, using a razor, gently split into two halves. After splitting, the gumball weighed 5.2289 g. Then, a STab-2 tablet weighing 0.3825 g was placed inside one half, and the gumball halves were rejoined by aligning the two halves and gently compressing by hand.

The gumball was then placed into a dry box. After nearly 19 hours, the gumball was removed from the dry box, opened, and the tablet was weighed at 0.3905 g. The tablet was then dissolved in 50.0 mL USP H₂O according to the test method listed above and a 10.0 mL aliquot was used to measure chlorine dioxide.

The following description is representative of Examples 16, 17, 30 and 31.

Example 16: A large round gumball (Dubble Bubble; Concord Confections, Ltd., Concord, Ontario, Canada) was placed onto a holder and, using a razor, gently split into two halves. After splitting, the gumball weighed 5.3388 g. Then, a STab-2 tablet weighing 0.3719 g was placed inside one half, and the gumball halves were rejoined by aligning the two halves and gently compressing by hand.

The gumball was then placed into a drawer under ambient conditions. After nearly 19 hours, the gumball was removed from the drawer, opened, and the tablet was weighed at 0.4002 g. The tablet was then dissolved in 50.0 mL USP H₂O according to the test method listed above and a 10.0 mL aliquot was used to measure chlorine dioxide.

The following description is representative of Examples 18, 19, 22, 23, 32 and 33. Note that Examples 22 and 23 used CSR1.05F powder, instead of STab-2 granules.

Example 19: A large round gumball (Dubble Bubble; Concord Confections, Ltd., Concord, Ontario, Canada) was placed onto a holder and, using a razor, gently split into two halves. After splitting, the gumball weighed 4.8773 g. Then, 0.4856 g of a STab-2 granules previously ground to <100 mesh was placed inside one half, and the gumball halves were rejoined by aligning the two halves and gently compressing by hand.

The gumball was then placed into a dry box. After nearly 19 hours, the gumball was removed from the dry box, opened, and the powder was weighed at 0.4744 g. The powder was then dissolved in 50.0 mL USP H₂O according to the test method listed above and a 10.0 mL aliquot was used to measure chlorine dioxide.

The following description is representative of Examples 20, 21, 34 and 35.

Example 20: A large round gumball (Dubble Bubble; Concord Confections, Ltd., Concord, Ontario, Canada) was placed onto a holder and, using a razor, gently split into two halves. After splitting, the gumball weighed 5.4447 g. Then, 0.4470 g of a STab-2 granules previously ground to <100 mesh was placed inside one half, and the gumball halves were rejoined by aligning the two halves and gently compressing by hand. The gumball was then placed into a drawer under ambient conditions. After nearly 19 hours, the gumball was removed from the drawer, opened, and the powder was weighed at 0.4650 g. The powder was then dissolved in 50.0 mL USP H₂O according to the test method listed above and a 10.0 mL aliquot was used to measure chlorine dioxide.

The following description is representative of Examples 24 and 37.

Example 24: A hollowed-out gum stick vessel (WM Wrigley Jr. Company, Chicago, Ill.) was created and weighed 8.5492 g. Then, three STab-2 tablets weighing a total of 1.1556 g was placed inside one half, and the vessel halves were rejoined by aligning the two halves and gently compressing by hand.

The gum vessel was then placed into a dry box. After nearly 18 hours, the gum vessel was removed from the dry box, opened, and the tablets were found to weigh a total of 1.1583 g. The tablets were then dissolved in 50.0 mL USP H₂O according to the test method listed above and a 10.0 mL aliquot was used to measure chlorine dioxide.

The following description is representative of Examples 25 and 36.

Example 25: A hollowed-out gum stick vessel (WM Wrigley Jr. Company, Chicago, Ill.) was created and weighed 9.0847 g. Then, three STab-2 tablets weighing a total of 1.1160 g was placed inside one half, and the vessel halves were rejoined by aligning the two halves and gently compressing by hand.

The gum vessel was then placed into a drawer under ambient conditions. After nearly 18 hours, the gum vessel was removed from the drawer, opened, and the tablets were found to weigh a total of 1.2834 g. The tablets were then dissolved in 50.0 mL USP H₂O according to the test method listed above and a 10.0 mL aliquot was used to measure chlorine dioxide.

The following description is representative of Examples 26 and 39.

Example 26: A hollowed-out gum stick vessel (WM Wrigley Jr. Company, Chicago, Ill.) was created and weighed 8.8663 g. Then, 1.1487 g of a STab-2 granules previously ground to <100 mesh was placed inside one half, and the vessel halves were rejoined by aligning the two halves and gently compressing by hand.

The gum vessel was then placed into a drawer under ambient conditions. After nearly 18 hours, the gum vessel was removed from the drawer, opened, and the powder was found to weigh a total of 1.2997 g. The powder was then dissolved in 50.0 mL USP H₂O according to the test method listed above and a 10.0 mL aliquot was used to measure chlorine dioxide.

The following description is representative of Examples 27 and 38.

Example 27: A hollowed-out gum stick vessel (WM Wrigley Jr. Company, Chicago, Ill.) was created and weighed 8.4739 g. Then, 1.1580 g of a STab-2 granules previously ground to <100 mesh was placed inside one half, and the vessel halves were rejoined by aligning the two halves and gently compressing by hand.

The gum vessel was then placed into a dry box. After nearly 18 hours, the gum vessel was removed from the dry box, opened, and the powder was found to weigh 1.1627 g. The powder was then dissolved in 50.0 mL USP H₂O according to the test method listed above and a 10.0 mL aliquot was used to measure chlorine dioxide.

C5-C8 were controls. C5 and C6 were each one-375 mg STab-2 tablets, and C7 and C8 were each 375 mg STab-2 granules, prepared fresh as described above.

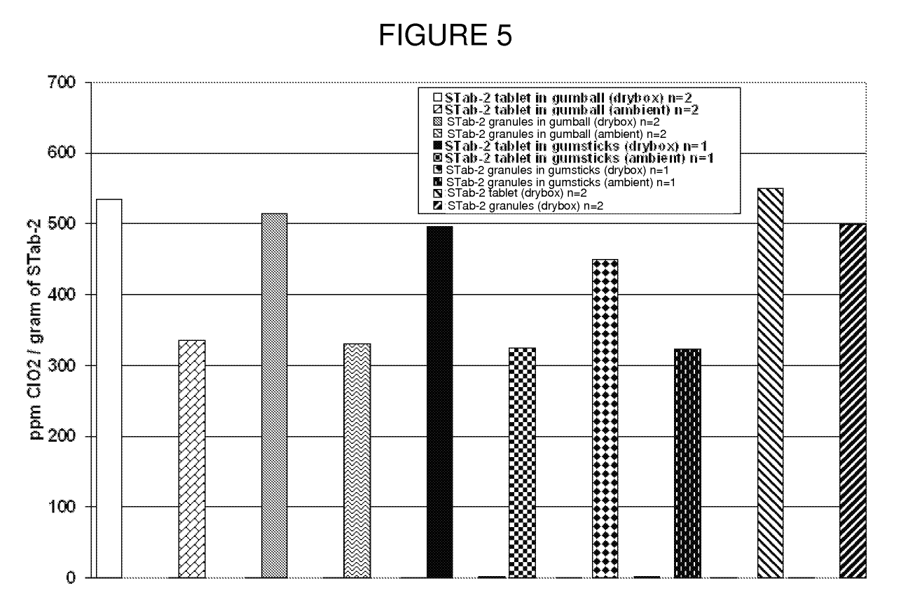

The data for the about 18-19 hour storage is presented in Table 2. The data are also presented as a bar graph in FIG. 5.

TABLE 2

| | | | | | | ppm ClO₂ |
|---|---|---|---|---|---|---|
| | | | | | | (ppm)/ |
| | | | | | | gram |
| | | | Deliery | Test | | Aseptrol |
| Ex. | Active | Storage | device | Method | ClO₂ | or |
| # | agent | method | form | used | (ppm) | CSR1.05F |
| 14 | STab-2 | Drybox | Gumball | Hach 2700 | 198 | 508 |
| 15 | STab-2 | Drybox | Gumball | Hach 2700 | 214 | 564 |
| 16 | STab-2 | Ambient | Gumball | Hach 2700 | 66 | 329 |
| 17 | STab-2 | Ambient | Gumball | Hach 2700 | 162 | 342 |
| 18 | STab-2 | Drybox | Gumball | Hach 2700 | 205 | 512 |
| | granules | | | | | |
| 19 | STab-2 | Drybox | Gumball | Hach 2700 | 206 | 517 |
| | granules | | | | | |
| 20 | STab-2 | Ambient | Gumball | Hach 2700 | 149 | 320 |
| | granules | | | | | |
| 21 | STab-2 | Ambient | Gumball | Hach 2700 | 116 | 342 |
| | granules | | | | | |
| 22 | CSR1.05F | Drybox | Gumball | titration | N/A | 515 |
| | powder | | | | | |
| 23 | CSR1.05F | Drybox | Gumball | titration | N/A | 655 |
| | powder | | | | | |
| 24 | STab-2 | Drybox | Gumstick | Hach 2700 | 575 | 496 |
| 25 | STab-2 | Ambient | Gumstick | Hach 2700 | 417 | 325 |
| 26 | STab-2 | Drybox | Gumstick | Hach 2700 | 584 | 450 |
| | granules | | | | | |
| 27 | STab-2 | Ambient | Gumstick | Hach 2700 | 375 | 323 |
| | granules | | | | | |
| C5 | STab-2 | n/a | n/a | Hach 2700 | 197 | 521 |
| C6 | STab-2 | n/a | n/a | Hach 2700 | 215 | 580 |
| C7 | STab-2 | n/a | n/a | Hach 2700 | 191 | 517 |
| | granules | | | | | |
| C8 | STab-2 | n/a | n/a | Hach 2700 | 181 | 482 |
| | granules | | | | | |

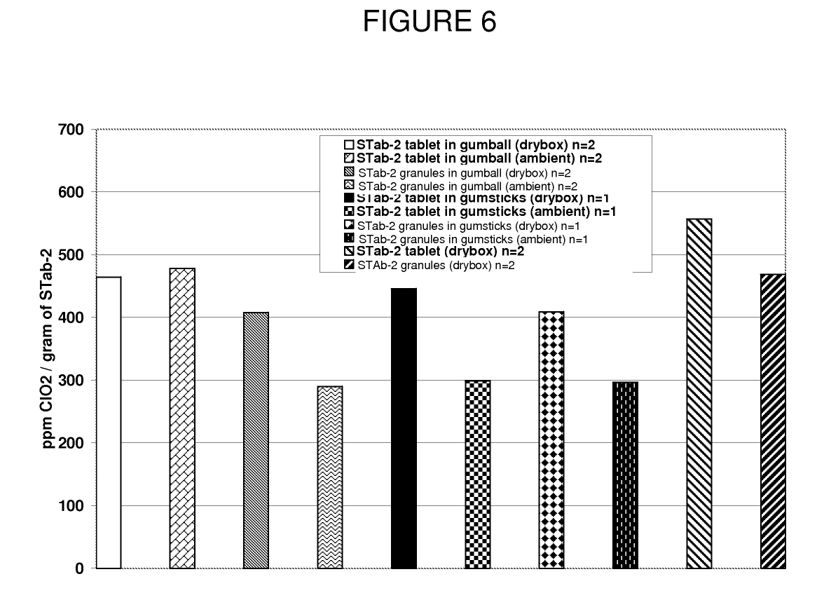

The data for the about 42 hour storage is presented in Table 3. The data are also presented as a bar graph in FIG. 6.

TABLE 3

| | | | Delivery | Test | | ppm |
|---|---|---|---|---|---|---|
| | Active | Storage | device | method | ClO₂ | ClO₂/gram |
| Ex. # | agent | method | form | used | (ppm) | Aseptrol |
| 28 | STab-2 | Drybox | Gumball | Hach 2700 | 172 | 425 |
| 29 | STab-2 | Drybox | Gumball | Hach 2700 | 196 | 503 |
| 30 | STab-2 | Ambient | Gumball | Hach 2700 | 209 | 502 |
| 31 | STab-2 | Ambient | Gumball | Hach 2700 | 208 | 454 |
| 32 | STab-2 | Drybox | Gumball | Hach 2700 | 167 | 418 |
| | granules | | | | | |
| 33 | STab-2 | Drybox | Gumball | Hach 2700 | 161 | 398 |
| | granules | | | | | |
| 34 | STab-2 | Ambient | Gumball | Hach 2700 | 98 | 267 |
| | granules | | | | | |
| 35 | STab-2 | Ambient | Gumball | Hach 2700 | 165 | 314 |
| | granules | | | | | |
| 36 | STab-2 | Ambient | Gumstick | Hach 2700 | 506 | 446 |
| 37 | STab-2 | Drybox | Gumstick | Hach 2700 | 344 | 299 |
| 38 | STab-2 | Drybox | Gumstick | Hach 2700 | 502 | 409 |
| | granules | | | | | |
| 39 | STab-2 | Ambient | Gumstick | Hach 2700 | 381 | 296 |
| | granules | | | | | |
| C9 | STab-2 | n/a | n/a | Hach 2700 | 215 | 562 |
| C10 | STab-2 | n/a | n/a | Hach 2700 | 207 | 551 |
| C11 | STab-2 | n/a | n/a | Hach 2700 | 174 | 460 |
| | granules | | | | | |
| C12 | STab-2 | n/a | n/a | Hach 2700 | 179 | 476 |
| | granules | | | | | |

These data clearly demonstrate that a substantial quantity of chlorine dioxide was produced by all of these embodiments. Storage in a drybox was associated with greater extent of chlorine dioxide-generating capacity. This result suggests that it is beneficial to store the oral care delivery device in such a way as to minimize exposure to ambient conditions. Nonetheless, both gumball embodiments and gumstick embodiments produced substantial amounts of chlorine dioxide after storage in ambient conditions. High retention of chlorine dioxide-generating capacity was observed for both the active agent as a tablet or large pieces of a tablet, and as a powder (<100 mesh). Thus, these studies demonstrate that oral care devices comprising chlorine dioxide-generating components, and in particular, a particular precursor of chlorine dioxide such as ASEPTROL® or CSR1.05F, can successfully generate chlorine dioxide after a period of storage and when the active agent is contacted with water.

### Experiment 3

### Active Agent is Chlorine Dioxide-Generating Components in Simulated Chewing

Additional embodiments were prepared as described below. To generally simulate chewing and exposure of the active agent to saliva, after storage each sample was placed in water, crushed, and chlorine dioxide generation was permitted for 15 minutes.

Examples 40-44 were formulated as gumballs.

Example 40: A large round gumball (Dubble Bubble; Concord Confections, Ltd., Concord, Ontario, Canada) was carefully rinsed with USP H₂O to remove any surface coloring agents, then placed onto a holder and, using a razor, gently split into two halves. After splitting, the gumball weighed 3.7591 g. Then, two STab-2 tablets weighing a total of 0.7475 g was placed inside one half, and the gumball halves were rejoined by aligning the two halves and gently compressing by hand.

The gumball was then placed into a dry box. After nearly 19 hours, the gumball was removed from the dry box, and was found to weigh a total of 4.5051 g. The entire gumball and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 41: A large round gumball (Dubble Bubble; Concord Confections, Ltd., Concord, Ontario, Canada) was carefully rinsed with USP H₂O to remove any surface coloring agents, then placed onto a holder and, using a razor, gently split into two halves. After splitting, the gumball weighed 4.6097 g. Then, one STab-2 tablet weighing 0.3597 g was placed inside one half, and the gumball halves were rejoined by aligning the two halves and gently compressing by hand.

The gumball was then placed into a dry box. After nearly 19 hours, the gumball was removed from the dry box, and was found to weigh a total of 4.9687 g. The entire gumball and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 42: A large round gumball (Dubble Bubble; Concord Confections, Ltd., Concord, Ontario, Canada) was carefully rinsed with USP H₂O to remove any surface coloring agents, which would interfere with the light absorption of the portable spectrophotometer, then placed onto a holder and, using a razor, gently split into two halves. After splitting, the gumball weighed 4.3155 g. Then, one STab-2 tablet weighing 0.3815 g was placed inside one half, and the gumball halves were rejoined by aligning the two halves and gently compressing by hand.

The gumball was then placed into a drawer under ambient conditions. After nearly 19 hours, the gumball was removed from the drawer, and was found to weigh a total of 4.7132 g. The entire gumball and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 43: A large round gumball (Dubble Bubble; Concord Confections, Ltd., Concord, Ontario, Canada) was carefully rinsed with USP H₂O to remove any surface coloring agents, then placed onto a holder and, using a razor, gently split into two halves. After splitting, the gumball weighed 3.3916 g. Then, 0.6572 g of a STab-2 granules previously ground to <100 mesh was placed inside one half, and the gumball halves were rejoined by aligning the two halves and gently compressing by hand.

The gumball was then placed into a dry box. After nearly 19 hours, the gumball was removed from the dry box, and was found to weigh a total of 4.0488 g. The entire gumball and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 44: A large round gumball (Dubble Bubble; Concord Confections, Ltd., Concord, Ontario, Canada) was carefully rinsed with USP H₂O to remove any surface coloring agents, then placed onto a holder and, using a razor, gently split into two halves. After splitting, the gumball weighed 4.1089 g. Then, 0.7182 g of a STab-2 granules previously ground to <100 mesh was placed inside one half, and the gumball halves were rejoined by aligning the two halves and gently compressing by hand.

The gumball was then placed into a drawer under ambient conditions. After nearly 19 hours, the gumball was removed from the drawer, and was found to weigh a total of 4.8437 g. The entire gumball and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

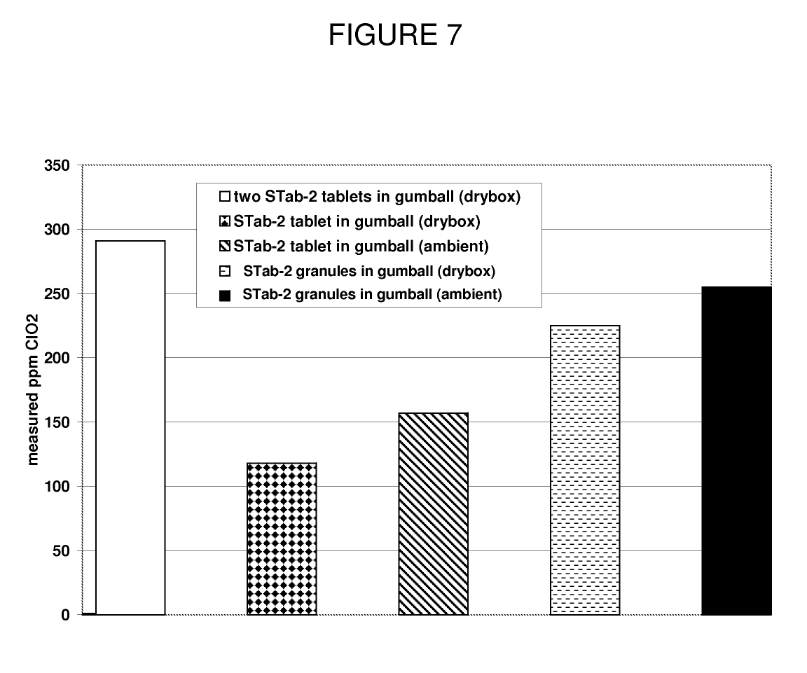

The data for Examples 40-44 is presented in Table 4, and as a bar graph in FIG. 7.

TABLE 4

| | | Storage | ClO₂ |
|---|---|---|---|
| Ex. # | Active agent | Method | (ppm) |
| 40 | STab-2 | Drybox | 291 |
| 41 | STab-2 | Drybox | 118 |
| 42 | STab-2 | Ambient | 157 |
| 43 | STab-2 granules | Drybox | 225 |
| 44 | STab-2 granules | Ambient | 255 |

Examples 45-49 were formulated as gumstick delivery devices.

Example 45:A hollowed-out gum stick vessel (Trident—Original Flavor; Cadbury Adams USA LLC., Parsippany, N.J.) comprising four chewing gum sticks was created (see FIG. 1) and weighed 5.6271 g. Then, two STab-2 tablets weighing a total of 0.7503 g was placed inside one half, and the vessel halves were rejoined by aligning the two halves and gently compressing by hand.

The gum vessel was then placed into a dry box. After nearly 23 hours, the gum stick vessel was removed from the dry box, and was found to weigh a total of 6.3582 g.

The entire gum stick vessel and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 46: A hollowed-out gum stick vessel (Trident—Original Flavor; Cadbury Adams USA LLC., Parsippany, N.J.) comprising four chewing gum sticks was created (see FIG. 1) and weighed 5.7355 g. Then, one STab-2 tablet weighing 0.3762 g was placed inside one half, and the vessel halves were rejoined by aligning the two halves and gently compressing by hand.

The gum vessel was then placed into a dry box. After nearly 23 hours, the gum stick vessel was removed from the dry box, and was found to weigh a total of 6.0861 g. The entire gum stick vessel and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 47: A hollowed-out gum stick vessel (Trident—Original Flavor; Cadbury Adams USA LLC., Parsippany, N.J.) comprising three chewing gum sticks was created (see FIG. 2) and weighed 4.7230 g. Then, one STab-2 tablet was split into two pieces, found to weigh a total of 0.3555 g, and both pieces of the tablet were placed inside one half of the gum stick vessel, and the vessel halves were rejoined by aligning the two halves and gently compressing by hand.

The gum vessel was then placed into a drawer under ambient conditions. After nearly 23 hours, the gum stick vessel was removed from the drawer, and was found to weigh a total of 5.1680 g. The entire gum stick vessel and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 48: A hollowed-out gum stick vessel (Trident—Original Flavor; Cadbury Adams USA LLC., Parsippany, N.J.) comprising three chewing gum sticks was created (see FIG. 2) and weighed 4.6778 g. Then, 0.2705 g of a STab-2 granules previously ground to <100 mesh was placed inside one half, and the vessel halves were rejoined by aligning the two halves and gently compressing by hand.

The gum vessel was then placed into a dry box. After nearly 23 hours, the gum stick vessel was removed from the dry box, and was found to weigh a total of 4.9280 g. The entire gum stick vessel and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

Example 49: A hollowed-out gum stick vessel (Trident—Original Flavor; Cadbury Adams USA LLC., Parsippany, N.J.) comprising three chewing gum sticks was created (see FIG. 2) and weighed 4.5958 g. Then, 0.3576 g of a STab-2 granules previously ground to <100 mesh (Notebook #30240-39-1) was placed inside one half, and the vessel halves were rejoined by aligning the two halves and gently compressing by hand.

The gum vessel was then placed into a dry box. After nearly 23 hours, the gum stick vessel was removed from the dry box, and was found to weigh a total of 5.0059 g. The entire gum stick vessel and its contents were then submerged in 50.0 mL USP H₂O and crushed with a pestle for 1 minute while submerged. After 15 minutes, a 10.0 mL aliquot was used to measure ppm of ClO₂ on a Hach DR 2700.

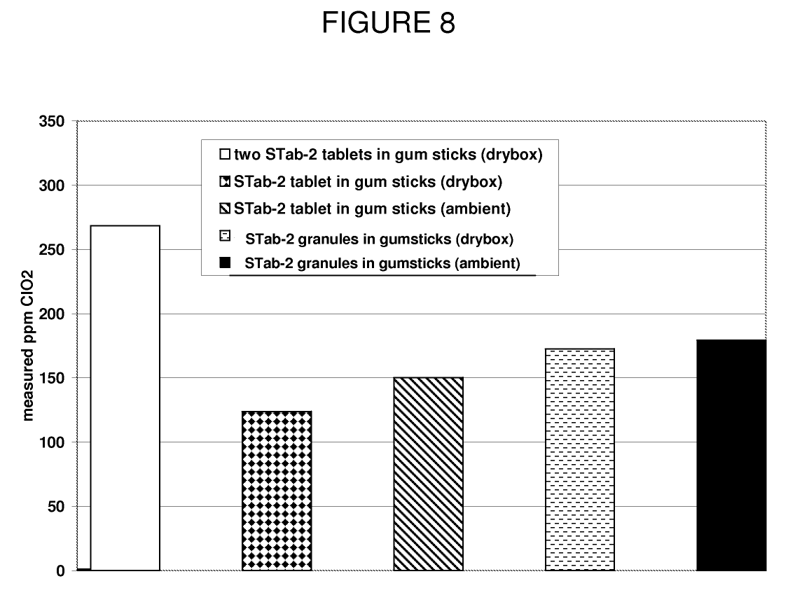

The data for Examples 45-49 is presented in Table 5 and as a graph in FIG. 8.

TABLE 5

| | | Storage | ClO2 |
|---|---|---|---|
| Ex. # | Active agent | Method | (ppm) |
| 45 | STab-2 | Drybox | 268 |
| 46 | STab-2 | Drybox | 124 |
| 47 | STab-2 (split in halves) | Ambient | 150 |
| 48 | STab-2 granules | Drybox | 173 |
| 49 | STab-2 granules | Ambient | 179 |

These data clearly demonstrate that a substantial quantity of chlorine dioxide is produced in the presence of the orally acceptable carrier and in a variety of embodiments. The gumball embodiments had slightly better chlorine dioxide production than the gumstick embodiments. Without wishing to be bound by theory, this result may relate to a lesser degree of reactivity of the gumball gum with chlorine dioxide versus the gumstick gum reactivity.

High retention of chlorine dioxide-generating capacity was again observed for both the active agent as a tablet or large pieces of a tablet, and as a powder (<100 mesh). Thus, these studies further demonstrate that oral care devices comprising chlorine dioxide-generating components, and in particular, a particular precursor of chlorine dioxide such as ASEPTROL, can successfully generate chlorine dioxide after a period of storage and when contacted with water. These oral care compositions and devices are a convenient and portable means for administering chlorine dioxide to the oral cavity.

The disclosures of each and every patent, patent application, and publication cited herein are hereby incorporated herein by reference in their entirety.

While the compositions, kits, and their methods of use have been disclosed with reference to specific embodiments, it is apparent that other embodiments and variations may be devised by others skilled in the art without departing from the true spirit and scope of the described compositions, kits and methods of use. The appended claims are intended to be construed to include all such embodiments and equivalent variations.

What is claimed is:

1. An oral care composition comprising:
a) an active agent selected from the group consisting of chlorine dioxide-generating components, a chlorine dioxide-containing solution, and combinations thereof; and
b) an orally acceptable carrier,
wherein the oral care composition provides an efficacious amount of chlorine dioxide in an oral cavity.

2. The composition of claim 1, wherein the active agent is compounded with the orally acceptable carrier.

3. The composition of claim 1, wherein the active agent is substantially encapsulated by the orally acceptable carrier.

4. The composition of claim 1, wherein the active agent is chlorine dioxide-generating components.

5. The composition of claim 4, wherein the active agent is a particulate precursor of chlorine dioxide.

6. The composition of claim 1, wherein the active agent is a chlorine dioxide-containing solution.

7. The composition of claim 6, wherein the chlorine dioxide-containing solution is a substantially pure chlorine dioxide solution.

8. The composition of claim 6, wherein the chlorine dioxide-containing solution is a thickened fluid.

9. The composition of claim 1, wherein the orally acceptable carrier comprises gum.

10. The composition of claim 1, wherein the active agent is coated with a wax coating.

11. An oral cavity delivery device comprising:
a first component comprising an active agent, the active agent being selected from the group consisting of chlorine dioxide-generating components, chlorine dioxide-containing solution, and combinations thereof; and
a second component comprising an orally acceptable carrier,
wherein the oral cavity delivery device can deliver an efficacious amount of chlorine dioxide in an oral cavity.

12. The device of claim 11, wherein the second component forms a container comprising at least one hollow cavity and wherein the at least one hollow cavity contains the first component and substantially encapsulates the first component therein.

13. The device of claim 12, wherein the container is substantially coated with a candy coating, a wax coating or both.

14. The device of claim 11, wherein the active agent is chlorine dioxide-generating components.

15. The device of claim 13, wherein the active agent is a particulate precursor of chlorine dioxide.

16. The device of claim 11, wherein the active agent is a chlorine dioxide-containing solution.

17. The device of claim 16, wherein the chlorine dioxide-containing solution is a substantially pure chlorine dioxide solution.

18. The device of claim 17, wherein the chlorine dioxide-containing solution is a thickened fluid.

19. The device of claim 11, wherein the orally acceptable carrier comprises gum.

20. The device of claim 11, wherein the first component is substantially encapsulated by a wax coating.
